(19) United States
(12) Patent Application Publication
(10) Pub. No.: US 2025/0135136 A1
(43) Pub. Date: May 1, 2025

(54) INSERTION TRACKING FOR ENDOLUMINAL INSTRUMENT

(71) Applicant: Covidien LP, Mansfield, MA (US)

(72) Inventors: Derek Scot Tata, Longmont, CO (US); Craig A. Pattan, Boulder, CO (US)

(73) Assignee: Covidien LP, Mansfield, MA (US)

(21) Appl. No.: 18/927,183

(22) Filed: Oct. 25, 2024

Related U.S. Application Data
(60) Provisional application No. 63/593,359, filed on Oct. 26, 2023.

(51) Int. Cl. A61M 16/04

(52) U.S. Cl. CPC A61M 16/0402; A61M 16/0488; A61B 1/267; A61M 16/0411; A61M 16/0497; A61M 2205/3306

(57) ABSTRACT

Methods and systems for an airway access device including a sensor that measures linear distance that an endoluminal tool has traveled into a patient. In an example, the airway access device includes a body; a lumen defined by the body; and an optical sensor, coupled to the body. The optical sensor includes an optical emitter that emits light towards an endoluminal instrument that passes through the lumen; an optical receiver that detects light reflected from the endoluminal instrument; and a processing unit that calculates a linear distance that the endoluminal instrument has traveled.

### CROSS-REFERENCE TO RELATED APPLICATIONS

This application claims the benefit of U.S. Provisional Application No. 63/593,359 filed Oct. 26, 2023, entitled “Insertion Tracking for Endoluminal Instrument,” which is incorporated herein by reference in its entirety.

### BACKGROUND

Endoluminal tools or instruments are commonly used to provide visualization of the airway and other anatomical lumens and cavities of a patient. One example of an endoluminal instrument is an endoscope, which is a narrow flexible tube that includes a camera and light source integrated into a steerable distal tip. During use, the distal tip of the endoscope may be inserted into the airway of a patient through an airway access device, such as an endotracheal tube (ETT) or other type of device. The endoscope may be inserted into an anatomical lumen to a specific depth, in order to avoid injury or damage to internal tissue. The insertion depth of the endoscope may also be used as a guide for positioning the ETT.

It is with respect to this general technical environment that aspects of the present technology disclosed herein have been contemplated. Furthermore, although a general environment is discussed, it should be understood that the examples described herein should not be limited to the general environment identified herein.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter. Additional aspects, features, and/or advantages of examples will be set forth in part in the description which follows and, in part, will be apparent from the description, or may be learned by practice of the disclosure.

In an aspect, the technology relates to an airway access device that includes a body; a lumen defined by the body; and an optical sensor, coupled to the body. The optical sensor includes an optical emitter that emits light towards an endoluminal instrument that passes through the lumen; an optical receiver that detects light reflected from the endoluminal instrument; and a processing unit that calculates a linear distance that the endoluminal instrument has traveled.

In an example, the airway access device is an endotracheal tube, the body defines a tubing connector, and the optical sensor is coupled to the tubing connector. In another example, the airway access device is an oropharyngeal airway, the body defines a bite block, and the optical sensor is coupled to the bite block. In still another example, the airway access device further includes a retaining guide that retains the endoluminal instrument in proximity with the optical sensor. In a further example, the retaining guide is a spring clip that is biased towards the optical sensor. In yet another example, calculating a linear distance that the endoluminal instrument has traveled includes comparing a first image captured by the optical receiver with a second image captured by the optical receiver to determine an incremental linear distance; and aggregating the linear distance with prior incremental linear distances to generate the aggregate linear distance that the endoluminal instrument has traveled. In still yet another example, the processing unit further causes the optical sensor to transmit the linear distance to an external device.

In another aspect, the technology relates to an airway access system that includes an endoluminal instrument controller that is coupled to an endoluminal instrument and an airway access sensor, coupled to an airway access device, in communication with the endoluminal instrument controller. The airway access sensor measures a linear distance that the endoluminal instrument passes by the airway access; and transmits the linear distance to the endoluminal instrument controller.

In an example, the endoluminal instrument controller is a video laryngoscope and the endoluminal instrument is one of an endoscope or an introducer. In another example, the airway access sensor is integrated into a tubing connector. In still another example, the endoluminal instrument comprises a motion sensor in a distal tip of the endoluminal instrument, and the endoluminal instrument controller receives motion data from the motion sensor; calculates a distance travelled by the endoluminal instrument based on the motion data from the motion sensor; compares the distance travelled to the linear distance from the airway access sensor; and based on the comparison, generating a notification.

In another aspect, the technology relates to a method for measuring a linear distance that an endoluminal instrument travels into a body. The method includes detecting, by an optical receiver of an airway access sensor, light reflected from the endoluminal instrument at multiple points in time; based on the detected light, calculating an incremental distance travelled by the endoluminal instrument for the multiple points in time; aggregating the incremental distances to form an aggregate linear distance; and displaying the aggregate linear distance as the endoluminal instrument travels into or out of the body.

In an example, the airway access sensor is coupled to an airway access device. In another example, the method further includes receiving motion data from a motion sensor in a distal tip of the endoluminal instrument; calculating a distance travelled by the endoluminal instrument based on the motion data; comparing the distance travelled based on the motion data with the aggregate linear distance; and based on the comparison, displaying a notification. In an example, calculating the incremental distances is performed by the airway access sensor and calculating a distance travelled by the endoluminal instrument based on the motion data is performed by an endoluminal instrument controller.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 depicts an example video system that includes a steerable endoscope.

FIG. 2A depicts an example endotracheal tube.

FIG. 2B depicts an example oropharyngeal airway.

FIGS. 2C-D depict views of the proximal end of the endotracheal tube of FIG. 2A.

FIG. 3 depicts an example optical sensor configured to detect linear movement of an endoluminal instrument.

FIG. 4 depicts an example method for determining linear insertion distance of an endoluminal instrument.

FIG. 5 depicts an example method for using motion data to verify insertion depth of an endoluminal instrument.

### DETAILED DESCRIPTION

Endoluminal tools or instruments are commonly used to perform, or to aid, any of a wide variety of medical procedures associated with anatomical lumens and cavities of a patient. One example of an endoluminal instrument is an endoscope, which is a narrow flexible tube that includes a camera, light source, and motion sensor integrated into a steerable distal tip. During use, the distal tip of the endoscope is inserted into the patient's body while the proximal end remains outside of the body. The proximal end is connected to a control device that allows a clinician to steer the distal tip and view video images acquired by the camera system. The distal tip of the endoscope may be navigated into an anatomical lumen or other cavity of a patient, such as the patient's airway, gastrointestinal (GI) tract, or another cavity. When navigated to a location of interest, the clinician may use the control device to articulate the steerable distal tip of the endoscope to establish a viewpoint of an anatomical structure or other biological matter.

In examples where the endoscope is used to visualize portions of a patient's airway, the endoscope may be inserted into the airway through an airway access device. An airway access device may be used to maintain patency of the upper airway (e.g., through the mouth and past the tongue), thereby providing a passage for breathing gases. In examples, an airway access device may include a lumen through which a ventilator may supply breathing gases to a patient, or through which the patient may breathe on their own. Examples of airway access devices include an endotracheal tube (ETT), oropharyngeal airway (OPA), laryngeal mask airway (LMA), and other types of breathing tubes and devices. To access the airway, the endoscope may be inserted through a proximal opening of the airway access device (e.g., at or near the mouth), through the lumen, and into the airway. When positioned in the airway, the endoscope may provide visualization of the larynx, carina, bronchi, and/or other features of the airway. In one example, the endoscope may be used to perform a bronchoscopy.

In other examples, an endoscope may be used to facilitate the insertion and positioning of an airway access device, such as during an intubation. An intubation is a procedure in which a clinician inserts an ETT through the mouth, past the larynx, and into the trachea of the patient. A laryngoscope may be used during intubation to help the clinician manipulate portions of the patient's anatomy, such as the tongue and epiglottis, and obtain a view of the larynx sufficient for inserting the ETT into the trachea. To further help visualize the larynx, some laryngoscopes may be configured with a video camera. A laryngoscope that includes a video camera may be referred to as a video laryngoscope (VL).

With some patients, the intubation may be difficult due to a variety of factors, such as inability to position the head or neck of the patient (e.g., due to injury), airway obstruction, atypical anatomy of the patient, and/or other factors. In these types of scenarios, clinicians may use an endoscope to augment the view provided by the VL, which may facilitate insertion of the ETT past the larynx. In some examples, the ETT is passed over the endoscope and into position in the airway, with the endoscope itself serving as a channel or guide for inserting the ETT. The process of navigating the endoscope into position then using the endoscope as a guide for the ETT may be referred to as post-loading. An endoscope used as a guide for breathing tube insertion may perform the same or similar functions as an introducer and may alternatively be referred to as an introducer in some examples.

Inserting and positioning the airway access device and navigating the endoscope within passages of the airway may pose risks to the patient. For example, with some patients, such as with pediatric patients, positioning the ETT in the trachea may be challenging due to the small size of the trachea. For instance, for some neonatal patients, the trachea may provide only a few millimeters in which the distal end of the ETT may be positioned between the larynx and carina. While the endoscope may provide or improve visualization of the larynx and/or trachea during intubation, the provided view may not be sufficient for determining how far to insert the ETT. An ETT that is inserted too far into the trachea may enter one of the bronchi, thereby restricting ventilation to only one of the lungs. In other examples, over-insertion of the ETT may cause damage to tissues or structures of the airway, such as to the trachea, carina, and/or other portions of the airway.

Further, navigating the endoscope within the airways may pose a risk of causing tissue damage. For example, the viewpoint of the endoscope camera may be obscured by biological matter (e.g., saliva, mucus, etc.) or may be obscured by a portion of the airway. In some examples, the distal tip of the endoscope may become lodged or stuck on an anatomical feature of the airway, which may both obscure the view provided by the endoscope camera and prevent further advancement of the distal tip through the airway. In such a situation, the clinician may continue to try to advance the endoscope, unaware of the condition of the endoscope tip. Due to the age or health condition of the patient, some tissues of the airway may be quite delicate and susceptible to damage from excessive force applied by the endoscope tip or other portions of the endoscope.

The technology described herein includes systems and methods for determining the linear insertion distance of an endoscope or other endoluminal instrument through an airway access device. The technology includes an optical sensor, such as an infrared (IR) sensor or other type of optical sensor, located at or near the proximal opening of the airway access device. The sensor may be connected to a control device associated with control of the endoscope (e.g., a VL) or another device suitable for receiving and displaying linear distance data provided by the sensor. For instance, the linear distance data may be displayed on a monitor associated with the control device (or other device) or may be used by an augmented visualization system (e.g., an augmented or mixed reality system) integrated with the control device to show the position of the endoscope in the body of the patient. As the endoscope is advanced into, or retracted from, the airway access device, the sensor may provide a measure of the total linear distance traveled by the endoscope, which may represent the insertion depth of the endoscope. In examples, the linear distance may be used to help a clinician position an ETT, such as during an intubation.

In further examples, the linear distance data may be combined with motion data acquired by one or more motion sensors located in the endoscope steerable tip. For instance, the endoscope steerable tip may include a motion sensor, such as a multi-axis accelerometer, inertial measurement unit (IMU), or other type of motion sensor. Discordance between motion sensor data and linear distance data may indicate a condition where the distal tip is stuck or otherwise unable to advance through the airway. A notification may be provided to the clinician, which may help reduce or prevent injury to tissues and/or structures of the airway.

In another example, the linear distance data may be combined with image data acquired by the endoscope camera to provide increased precision of endoscope position control. For instance, during an initial navigation of the endoscope through the airway, the image data acquired by the endoscope camera may be used to perform image recognition of anatomical structures and/or regions within the airway (e.g., trachea, carina, first generation of the bronchial tree, etc.). During subsequent navigation of the endoscope through the airway, the image data may be used to coarsely identify the general region of the airway (or other segment of the body) and the linear distance data may be used to provide more precise position information within the identified region. In one example, the combination of endoscope image data and linear distance data may be used to help the clinician navigate the endoscope to a particular area of interest in a consistent and repeatable way. For example, the combined data may be used by the clinician to navigate the endoscope to a particular area, feature, nodule, etc., to observe the effect of a medical procedure or treatment over time. For instance, the linear distance data from the sensor discussed herein may be used in combination with the image recognition data to display a position of an endoscope within the body, such as in an augmented reality display. One example of suitable augmented reality display is discussed in U.S. Publication No. 2021/0137350 (hereinafter the ‘350 Publication), titled Steerable Endoscope System with Augmented View, which is incorporated herein by reference in its entirety.

Additional details are now provided by way of discussion of the included drawings.

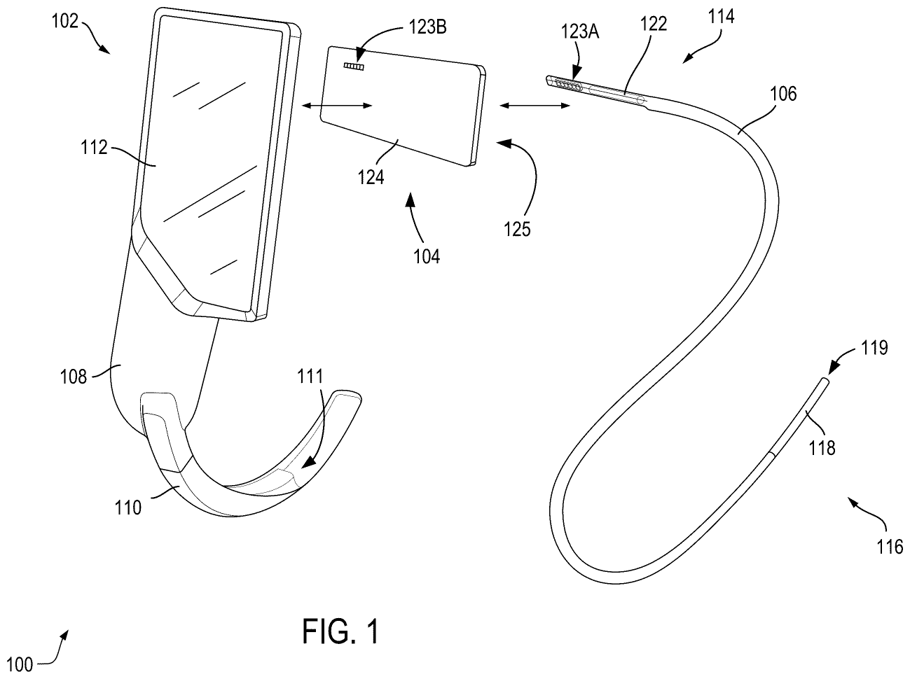

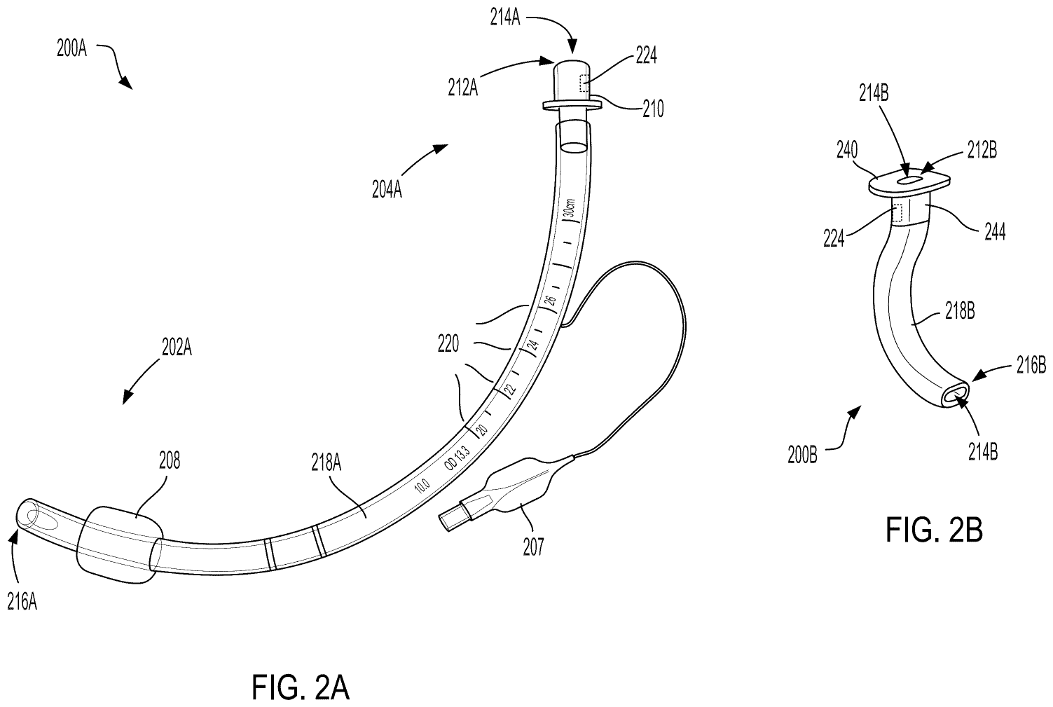

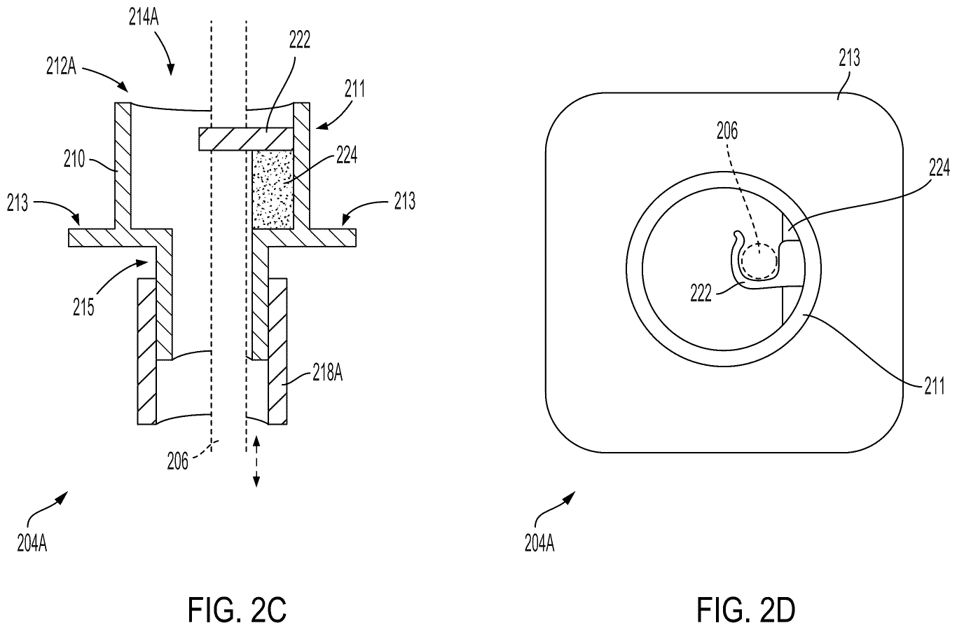

FIG. 1 depicts an example medical video system 100 that includes a video laryngoscope (VL) 102 and steerable endoscope 106. The system 100 may be used to insert and position an ETT during an intubation, such as described above. The VL 102 is capable of connecting to, and providing steering control of, the endoscope 106, through a detachable cartridge 104. The endoscope 106 is one example of an endoluminal instrument that may be used in conjunction with an airway access device and airway access sensor, such as depicted in FIGS. 2A-D and described below. The VL 102 similarly is one example of an endoluminal instrument controller.

As discussed further herein, the VL 102 may be in communication with an airway access sensor that measures the linear distance that the endoluminal instrument travels past the sensor (e.g., into the body of a patient). The linear distance may then be displayed on the VL 102. The VL 102 may similarly perform its own operations to determine the position of the endoluminal instrument based on motion data received from a motion sensor in the endoluminal instrument. These two measurements of position and/or distance traveled may be compared or otherwise used in determining insertion depth and/or blockages or tissue that is preventing the endoluminal instrument from further progressing into the body.

As further detail, the distal end 116 of the endoscope 106 includes a steerable tip 118 and accessories 119, which may be used during operation of the endoscope 106. The accessories 119 may include a camera system (e.g., a video camera, lights, etc.) that captures image data (e.g., video images of the airway) during use. The accessories 119 may also include sensors, such as an accelerometer or IMU, which provides motion data associated with the acceleration, angular velocity, position, and/or other variables associated with the position/orientation/movement of the steerable tip 118. In some examples, the accessories 119 may further include one or more instrument ports, such as a port for a working channel (not depicted).

The steerable tip 118 is connected to a drive system 122 by one or more pairs of pull wires (not depicted), which are routed along the interior of the endoscope 106 from the drive system 122 to the endoscope distal end 116. The drive system 122 applies steering forces to the pull wires, which causes articulation of the endoscope steerable tip 118.

The endoscope proximal end 114 also includes an electrical interface 123A, through which the endoscope 106 may receive electrical power and may transmit/receive signals to/from the VL 102. For example, the electrical interface 123A may provide power and/or steering control signals from the VL 102 to the drive system 122 for controlling the movement of the steerable tip 118. The electrical interface 123A also provides a source of input power for operating the accessories 119 (such as the camera system, IMU, etc.), and/or other sensors or electronic elements included within the endoscope 106.

Further, the electrical interface 123A provides a data path for transmitting IMU motion data, video image data, and/or other types of data from the endoscope 106 to the VL 102. In some examples, signals or data (such as clock, enable, timing, and/or other signals) may be transmitted/received through the electrical interface 123A in order to enable or configure operation of the endoscope 106.

The electrical interface 123A may include a plurality of electrical contacts, such as conductive pads, receptacles, pins, balls, ports, and/or other type of electrical contacts that are connected to elements of the endoscope 106 by a plurality of conductors routed within the interior of the endoscope 106. The conductors (not depicted) may include one or more electrical wires, flexible printed circuits (FPCs), electrical cables, and/or other types of electrical conductors suitable for distributing power and establishing signal connection between the electrical interface 123A and elements of the endoscope 106. For instance, wires may be routed along the internal length of the endoscope 106 between the electrical interface 123A and the accessories 119 (e.g., the endoscope camera system, IMU, etc.). The electrical wires provide power and signal connectivity to the accessories 119.

To connect the endoscope 106 to the VL 102, the endoscope proximal end 114 is connected to the detachable cartridge 104, which serves as an electrical and/or mechanical interface between the VL 102 and endoscope 106. In other examples, the endoscope 106 may connect to the VL 102 by another type of cartridge 104, or the endoscope 106 may connect directly to the VL 102, such as at a connection port included with the VL 102.

The cartridge 104 further includes an electrical interface 123B on the cartridge rear surface 124, for making electrical connection with the VL 102. Within the cartridge 104, the electrical interface 123B is connected to an electrical interface on the cartridge front surface 125 (not depicted), such as by wiring, pins, printed circuit board (PCB), flex, and/or other type of electrical connection. The electrical interface on the cartridge front surface 125 includes conductive elements (e.g., pads, pins, etc.) that make electrical contact with corresponding elements of the endoscope electrical interface 123A, when the endoscope 106 is connected to the cartridge 104.

During a post-loading procedure, the endoscope proximal end 114 is disconnected from the cartridge 104 and an ETT is slid over the endoscope 106. The proximal end 114 may then be reconnected to the cartridge 104, so that video image data and IMU data may be received by the VL 102, and so that the steerable tip 118 may again be controlled through the VL 102.

The VL 102 includes a display 112, a handle 108, and a blade or extension 110, which includes a camera 111 positioned near the distal end of the extension 110. The VL 102 may include additional functions, features, and/or elements typically associated with a video laryngoscope, such as a power source (e.g., a battery), processor, memory, and other electronic components. The VL 102 may further include elements associated with a wireless communication feature. For example, the VL 102 may be capable of communicating with other devices using a wireless communication technology, such as Bluetooth, WiFi, and/or other wireless technologies. As an example, the VL 102 may connect to an airway access sensor (depicted in FIGS. 2B-C and 3) via Bluetooth. In other examples, the VL 102 may be connected to an airway access sensor and/or other sensors and devices via wired connection, such as through a port provided by the VL 102.

In an example, the VL 102 receives data (such as video images and sensor data) from the steerable endoscope 106 through the cartridge 104 and displays the received data on the display 112. The display may be capable of displaying images from multiple cameras simultaneously, such as images from the VL camera 111 and the endoscope camera, such as by split screen, picture-in-picture, or other display methods. The display 112 may be any of a variety of display technologies, such as LCD, LED, OLED, or other display technology. In examples, the display 112 may be a touch-sensitive display (e.g., a capacitive touch-sensitive display) that allows the user to provide steering input through the display 112. Elements of the VL 102 may translate the steering inputs to corresponding motor outputs for articulating the endoscope steerable tip 118. The VL 102 may further receive data from an airway access sensor and display the total linear insertion distance of the endoscope 106 through an airway access device, as described herein.

Additionally or alternatively, the endoscope 106 may be connected to other types of control devices capable of receiving the endoscope proximal end and establishing mechanical and/or electrical connection with the endoscope 106. In examples, the control device may provide steering control of the endoscope steerable tip 118 and may receive and display video image data from the endoscope 106.

In additional examples, the endoscope 106 and/or VL 102 may be associated with an augmented visualization system (not depicted). The augmented visualization system may include one or more devices (e.g., a headset, an augmented visualization display, etc.) that allows the clinician to view multiple types of data in an integrated viewing format. For example, the augmented visualization system may provide a type of display that allows the clinician to view the position of the endoscope in the body of the patient as a visualization overlay on the patient, as described in the ‘350 Publication. The position of the endoscope 106 displayed in the augmented reality view may be based on the linear distance calculations discussed herein.

FIG. 2A depicts an example airway access device 200A, which in this example is a type of ETT. When positioned in the airway of a patient, the ETT 200A may be used to pass breathing gases between the patient and a ventilator. As described above, during intubation the ETT distal end 202A is inserted through the mouth, past the larynx, and into the trachea of the patient. The ETT body 218A is a flexible tube that allows the ETT 200A to bend and flex as the ETT 200A is navigated into position. In some examples, the ETT body 218A may be shaped, such as with the curve depicted, to match the approximate shape of the airway, which may facilitate insertion of the ETT 200A. The ETT body 218A may include depth markings 220 that provide an external visual indication of the insertion depth of the ETT 200A. When positioned, a pilot balloon 207 may be used to inflate the cuff 208, which seals the ETT 200A against the walls of the trachea. The cuff 208 is shown inflated in FIG. 2A, but in some examples, the cuff 208 may remain uninflated during use.

The ETT proximal end 204A remains outside the patient and is connected to a ventilator through tubing (e.g., a patient circuit), which attaches to the ETT 200A at a tubing connector 210. In examples, the tubing connector 210 may be a 15 mm connector or other suitable connector for attaching the patient circuit. When connected to the patient circuit, inhaled breathing gases may be supplied by the ventilator to the patient through the ETT 200A. In examples where the cuff 208 is inflated, exhaled breathing gases may be vented through the ETT 200A.

In one example, the distal end of an endoscope (e.g., distal end 116) or other type of endoluminal instrument may be inserted into the proximal opening 212A of the tubing connector 210 and advanced through the lumen 214A of the ETT 200A. At the distal end 202A of the ETT 200A, the distal tip of the endoscope may be advanced through the distal opening 216A and into the airway. The endoscope may then provide video images of the airway, such as the trachea, carina, bronchi, and/or other structures of the airway and/or lungs of the patient.

In some examples, the tubing connector 210 may include an airway access sensor 224 that measures the linear insertion distance of the endoscope as it is advanced through the ETT 200A. In other examples, the sensor 224 may be detachable from the tubing connector 210 and may be attached to the tubing connector 210 prior to inserting the endoscope. In further examples, the sensor 224 may be attached to another other portion of the ETT proximal end 204A near the proximal opening 212A. An example location and configuration for the sensor 224 is depicted in FIGS. 2C-D and described below.

In another example, and as noted above, the endoscope may be used to aid insertion and positioning of the ETT 200A during an intubation. In such an example, the endoscope may be navigated into position in the airway and used as a guide (e.g., an introducer) for positioning a post-loaded ETT 200A. The ETT distal end 202A may be slid over the proximal end of the endoscope (e.g., proximal end 114) and, using the endoscope to visualize the airway, the ETT 200A may be positioned. Following placement of the ETT 200A, the endoscope may be retracted through the ETT proximal end 204A. An airway access sensor 224 associated with the tubing connector 210 may be used to track the linear distance traveled by the endoscope as it is retracted from the ETT 200A. The distance may be used for additional procedures, such as for further exploration of the airway, to reposition the ETT 200A, and/or for another purpose.

While the airway access sensor 224 is depicted as being integrated into the connector 210 that is part of the ETT 200A, in other examples, the airway access sensor 224 my be integrated into a standalone tubing connector or other connector that may be coupled and decoupled (e.g., connected and disconnected) to different types of tubing and/or airway access devices. Accordingly, the airway access sensor and the connector may be used with many different kinds of airway access devices. In some examples, the sensor and/or connector may also be reused.

FIG. 2B depicts another example airway access device 200B, which in this example is a type of OPA. The OPA 200B is inserted through the mouth, past the tongue, and into the pharynx of the patient. The OPA 200B is used to maintain patency of the upper airway, such as when a patient is sedated (or at other times). When the OPA 200B is fully inserted into the mouth, the bite block 244 is approximately aligned with or may be in contact with the patient's teeth. The bite block 244 is designed to prevent the patient's mouth from fully closing, which would occlude the airway and potentially damage an endoscope or other endoluminal instrument that may be present. The mouth flange 204 remains outside of the mouth and may rest against or be near the patient's lips. The mouth flange 204 prevents or reduces the risk of the OPA 200B being inserted too far into the upper airway. The OPA body 218B may be curved, as shown, to approximately conform to the shape of the tongue and/or portions of the pharynx. Further, the OPA body 218B may be rigid or semi-rigid in order to prevent the tongue from blocking the airway.

In the example depicted, the OPA 200B includes a lumen 214B that passes through the OPA body 218B and connects a proximal opening 212B to a distal opening 216B. The lumen 214B provides an additional passage for the flow of inhaled and exhaled breathing gases through the OPA 200B. The lumen 214B also provides airway access to an endoscope, as such described above for the ETT 200A. The endoscope may be inserted through the proximal opening 212B, advanced through the lumen 214B, and extended into the airway through the distal opening 216B. Because the OPA 200B extends into the pharynx, rather than past the larynx (such as with the ETT 200A), an endoscope inserted through the OPA 200B may provide a view of the larynx and associated airway features.

As described herein, the OPA 200B may include an airway access sensor 224 that provides data associated with the linear insertion distance of the endoscope. In some examples, the airway access sensor 224 may be included near the proximal opening 212B. For instance, the sensor 224 may be included as part of the mouth flange 240 and/or bite block 244. In other examples, the sensor may be detachable from the OPA 200B, and may be attached prior to insertion of the endoscope.

In still other examples, other types of airway access devices may be used to maintain airway patency, provide for the passage of inhaled/exhaled breathing gases, and/or perform other respiratory functions. Such airway devices may include a sensor that provides data associated with the linear insertion distance of an endoscope.

FIG. 2C depicts an enhanced cross-sectional view of the ETT proximal end 204A, and FIG. 2D depicts an enhanced top view of the ETT proximal end 204A. The tubing connector 210 includes a tubing port 211, tubing flange 213, and nozzle 215. As briefly noted above, the tubing port 211 may be a 15 mm port that connects to the tubing associated with a patient circuit. In other examples, the tubing port 211 may be larger or smaller than 15 mm, according to the patient circuit being connected. The tubing flange 213 provides a mechanical stop for the patient circuit tubing and for tubing of the ETT body 218A, and may serve as a type of handle for manipulating the tubing connector 210. In some examples, the ETT body 218A may be permanently connected to the nozzle 215, such as by adhesive, thermal bonding, or by other method of fixation. In other examples, the tubing connector 210 maybe detachable from the ETT body 218A.

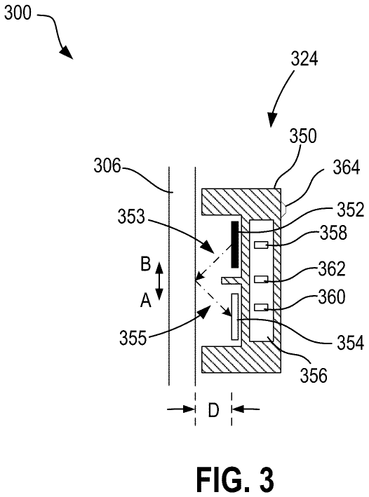

The tubing connector 210 includes an optical sensor 224, positioned adjacent to and/or partially within the wall of the tubing port 211. The sensor 224 may include an optical emitter that transmits light onto an inserted endoluminal instrument 206. The sensor 224 may further include an optical receiver that receives or detects light reflected from the endoluminal instrument 206. The sensor 224 acquires linear distance data associated with advancement/retraction of the endoluminal instrument 206 into/out of the airway. Additional details are provided below with respect to FIG. 3.

In some examples, the inserted endoluminal instrument 206 may be an endoscope, such as described above. In other examples the endoluminal instrument 206 may be any of a wide variety of instruments suitable for insertion into tubing connector 210. For example, the endoluminal instrument 206 may be a type of endoscope, bougie, introducer, stylet, guide, and any other type of endoluminal instrument that may be suitably inserted into the tubing connector 210.

In the example depicted, the tubing connector 210 further includes a retaining guide 222 that retains the endoluminal instrument 206 in close proximity to the sensor 224. For instance, in some examples, the optical sensor 224 functions better (e.g., is more accurate) when the endoluminal instrument 206 is closer to the sensor 224 (e.g., in contact with or nearly in contact with the sensor 224). The retaining guide 222 may be positioned on the interior of the tubing port 211 so as to prevent interference between the tubing port 211 and an associated patient circuit. Further, the retaining guide 222 may be positioned near the ETT proximal opening 212A to improve clinician access to the retaining guide 222. In examples, the retaining guide 222 may be formed as part of the tubing port 211 (e.g., through a molding or extrusion process). For instance, the retaining guide 222 may be of the same material as, and continuous with, the tubing port 211 or other portions of the tubing connector 210. In other examples, the retaining guide 222 may be separate from the tubing port 211 and may be affixed to the interior of the tubing port 211.

The retaining guide 222 may present a relatively low-friction surface to the endoluminal instrument 206, such as to allow the endoluminal instrument 206 to be freely advanced/retracted through the retaining guide 222. The top view of the ETT proximal end 204A depicted in FIG. 2D may represent the view of a clinician when inserting the endoluminal instrument 206 into the tubing connector 210. The clinician inserts the endoluminal instrument 206 into the tubing port 211 and into the retaining guide 222 and may then advance the endoluminal instrument 206 further into the ETT body 218A. The retaining guide 222 may include features or elements that facilitate rapid insertion of the endoluminal instrument 206. For example, the retaining guide 222 may include edges, chamfers, and/or other features that aid insertion of the endoluminal instrument 206 into the retaining guide 222. When the endoscopic procedure is complete, the endoluminal instrument 206 may be retracted from the retaining guide 222 and withdrawn from the ETT proximal opening 212A.

In some examples, the retaining guide 222 may be formed as spring clip that is biased towards a closed position. The spring clip may be a C-clip or other type of clip that when the endoluminal instrument 206 is inserted through the opening of the spring clip, the spring clip pushes the endoluminal instrument 206 towards the optical sensor 224.

FIGS. 2C-D depict one example arrangement in which the sensor 224 and retaining guide 222 are positioned adjacent to one another. In other examples, the retaining guide 222 and sensor 224 may be located and/or positioned within the tubing connector 210 in any arrangement that allows the retaining guide 222 to retain the endoluminal instrument 206 in close proximity to the sensor 224 during use. As an example, the sensor 224 may be positioned within a portion of the nozzle 215, or other portion of the tubing connector 210, and the retaining guide 222 may remain near the ETT proximal opening 212A.

In addition, the retaining guide 222 is depicted as including a single retaining element that retains the endoluminal instrument 206 near the sensor 224. In other examples, the retaining guide 222 may include two or more elements that retain the endoluminal instrument 206 near the sensor 224. Further, the retaining guide 222 may be configured or shaped differently than depicted in FIGS. 2C-D. For example, the retaining guide 222 may designed as one or more loops, rings, tracks, guides, lumens, and/or other elements suitable for retaining the endoluminal instrument 206 in proximity to the sensor 224.

In still other examples, the sensor 224 and retaining guide 222 may be implemented together in a separate component from the tubing connector 210. For instance, the sensor 224 and retaining guide 222 may be incorporated into an additional or separate connector or adapter that may be connected between the nozzle 215 and ETT body 218A or between the tubing connector 210 and the ventilation tubing from the ventilator. In such an example, the retaining guide 222 may be designed accordingly, to retain the endoluminal instrument 206 near the sensor 224 as the endoluminal instrument 206 is inserted and advanced into the ETT body 218A. In another example, the sensor 224 and retaining guide 222 may be incorporated into a connector or adapter that connects to the tubing port 211.

In additional examples, the sensor 224 may be included as part of the tubing connector 210 and the retaining guide 222 may be included as a separate component. For example, prior to insertion of the endoluminal instrument 206, an external retaining guide 222 may be attached to a portion of the tubing connector 210, such as the tubing port 211, nozzle 215, or other portion of the tubing connector 210 and/or ETT body 218A.

FIG. 3 depicts an example sensing configuration 300 that includes an endoluminal instrument 306 adjacent to an optical sensor 324. The sensor 324 may be similar to, or the same as, optical sensor 224 depicted in FIGS. 2A-D and described above. The sensing configuration 300 may be associated with a retaining guide (e.g., retaining guide 222) or similar feature that maintains the distance D between the endoluminal instrument 306 and the optical components of the sensor 324. As noted above, the endoluminal instrument 306 may be any of a wide variety of instruments suitable for insertion into the airway of a patient through an airway access device. For example, the endoluminal instrument 306 may be an endoscope, bougie, introducer, etc.

The sensor 324 includes an optical emitter 352 that serves as a source of transmitted light 353. The transmitted light 353 may be infrared (IR) or a visible wavelength of light (e.g., red light). The emitter 352 may include one or more light emitting diodes (LEDs) and/or other types of light sources capable of emitting light in the IR spectrum or visible spectrum. The transmitted light 353 reflects from the surface of the endoluminal instrument 306 and impinges on an optical receiver 354 as reflected light 355. The optical receiver 352 detects the reflected light 355 and transduces the optical of the reflected light into an electrical signal, which may then be processed by the sensor and/or another device (e.g., video laryngoscope, controller).

Larger distances D between the optical emitter 352 and the endoluminal instrument 306 may reduce the intensity of reflected light 355 received by the optical receiver 354. In addition, larger distances D may result in the reflected light 355 diverging away from the optical receiver, potentially reducing the ability of the optical receiver 354 to accurately detect movement of the endoluminal instrument 306. Thus, the position of the endoluminal instrument 306 is maintained in close proximity to the sensor 324. In examples, the distance D between the endoluminal instrument 306 and the surfaces of the optical emitter 352 and optical receiver 354 may be at least 0.1 mm, and less than 1 mm or less than 2 mm. In an airway access device (e.g., ETT 200A), one or more retaining guides or other retaining elements may be used to maintain the distance D, such as depicted in FIGS. 2C-D.

In addition, the optical emitter 352 and optical receiver 354 are depicted in FIG. 3 as being substantially co-planar. In other examples, the optical emitter 352 and optical receiver may be arranged, oriented, and/or positioned differently. For example, the optical emitter 352 and optical receiver 354 may be arranged at an angle to one another or may be positioned in another arrangement, so that reflected light 355 may be suitably received by the optical receiver 354.

The optical receiver 354 may include any of a variety of optical sensing elements. For example, the optical receiver 354 may include or be a type of one or more photodiodes, complementary metal-oxide semiconductor (CMOS) sensor, charge-coupled device (CCD) sensor, or other type of sensor that captures the reflected light 355 as a series of images. In some examples, the optical receiver 354 is capable of detecting the reflected light 355 at a sampling rate of at least 100 Hz. In further examples, the optical receiver 354 may function as a type of IR camera or other type of camera that images the endoluminal instrument 306 at an image frame rate. For instance, the optical sensor 354 may be capable of providing images at more than 500 image frames per second, more than 1000 image frames per second, or at another image frame rate. As the endoluminal instrument 306 is advanced in direction A or retracted in direction B, surface features of the endoluminal instrument 306 cause changes in the reflected light 355 that are discernible to the sensor 324 based on the detections from the optical receiver 354. The image frames captured by the optical receiver 354 may include small differences in the imaged surface of the endoluminal instrument 306 and these differences may be used to determine direction and distance travelled by the endoluminal instrument 306.

In other examples, the optical receiver 354 may include other types of sensing elements, such as one or more photodiodes, phototransistors, and/or other types of photo-sensitive elements suitable for discerning movement of the endoluminal instrument 306.

The optical emitter 352 and/or optical receiver 354 may be connected to a circuit board 356 included as part of the sensor 324. In examples, the circuit board 356 may include elements that provide electrical power to the optical emitter 352 and/or that control the intensity, frequency, and/or modulation of transmitted light 353 generated by the optical emitter 352. For example, the circuit board 356 may include elements that control the amount of electrical current provided to LEDs of the optical emitter 352. In other examples, the circuit board 356 may include elements that control other aspects of the operation of the optical emitter 352.

The circuit board 356 may further include elements that receive and process signals generated by the optical receiver 354 in response to the received light 355. In examples where the optical receiver 354 captures image frames of the reflected light 355 (as described above), the circuit board 356 may include one or more processing units or elements 358 that process/analyze the received image frames. For example, the circuit board 356 may include a digital signal processor (DSP), field-programmable gate array (FPGA), application-specific integrated circuit (ASIC), microprocessor, microcontroller, and/or other processing elements capable of analyzing image frames and determining the direction and distance travelled by the endoluminal instrument 306.

In some examples, processing the image frame data includes applying digital filtering and/or amplification, using one or more signal processing algorithms, and/or performing other types of signal or image processing. In one example, a processing element of the circuit board 356 identifies one or more surface features of the endoluminal instrument 306 in one or more of the received image frames. For instance, as the endoluminal instrument 306 is advanced in direction A the processing element may analyze one or more subsequent image frames and determine that the surface features have shifted in direction A and may further determine the distance that the surface features have traveled. Similarly, image frames acquired by the optical receiver 354 may include surface features of the endoluminal instrument 306 that shift in direction B between image frames. A processing element of the circuit board 356 may identify this shift and further determine the distance the surface features have traveled. In this regard, the image frames acquired by the optical receiver 354 may be used to determine direction and distance travelled by the endoluminal instrument 306.

The circuit board 356 may further analog and/or digital circuit elements for processing received signals. For example, the circuit board 356 may include passive components, amplifiers, filters, analog-to-digital converters (ADCs), digital logic, and/or other types of circuit elements. In some examples, the circuit board 356 may include one or more memory elements 360 for storing image frame data provided by the optical receiver 354, processed data, and/or other types of data.

The processing element(s) 358 of the circuit board 356 may also provide for the control and/or function of the optical receiver 354. For instance, elements of the circuit board 356 may provide for the control of the image frame rate, image resolution, and/or other aspects of the operation of the optical receiver 354. In further examples, the circuit board 356 may receive or provide signals associated with the control and/or operation of the optical emitter 352 and/or optical receiver 354. In examples, the circuit board 356 may receive or provide clock, timing, enable, and/or other signals to/from the optical emitter 352 and/or optical receiver 354.

The circuit board 356 may include one or more printed circuit boards (PCBs), flexible printed circuits (FPCs), or other type of circuit board(s). In some examples, the optical emitter 352 and/or optical receiver 354 may be connected to the circuit board 356 via one or more electrical conductors, such as cables, wires, etc. In other examples, the optical emitter 352 and/or optical receiver 354 may be included as part of the circuit board 356.

In the example depicted in FIG. 3, the circuit board 356, optical emitter 352, and optical receiver 354 are depicted as being included in a single housing 350. In other examples, the circuit board 356, optical emitter 352, and/or optical receiver 354 may be part of two or more separate housings 350. For instance, the optical emitter 352 and optical receiver 354 may be included together in a first housing and the circuit board 356 may be included in a second housing, which is electrically connected to the first housing (such as by one or more wires, cables, etc.).

In addition, the circuit board 356 may also include one or more communication interfaces 362 (e.g., wireless or wired communication interfaces) that allow for the sensor 324 to communicate with one or more external devices. For example, the sensor 324 may be connected to an external device, such as an external monitor, controller, and/or a control device used to provide steering control of a steerable endoscope. As an example, the sensor 324 may be in communication with the video laryngoscope, which may display the linear distances discussed herein. Alternatively, the sensor 324 may be connected to other types of devices capable of receiving and displaying linear distance data, such as a computer, other medical device, or other type of device. The external device may be connected to the sensor 324 by one or more conductors, wires, cables, or other connection method (not depicted), which may be connected within the sensor 324 to the circuit board 356, optical emitter 352, and/or optical receiver 354. The connection may be used by the external device to provide power to the sensor 324, and to transmit/receive signals to/from the sensor 324. For example, the external device may configure the sensor for operation, such as by providing signals that control the intensity of transmitted light 353, the image frame rate, image resolution, sampling rates, filtering, and/or a range of other configurable parameters of the sensor 324. In some examples, the external device may provide configuration and control data to elements of the circuit board 356, which in turn directly controls aspects of the operation of the optical emitter 352 and/or optical receiver 354.

The sensor 324 may also provide or receive signals associated with the operation of the sensor 324. For example, the sensor 324 may provide or receive signals associated with communication between the sensor 324 and external device, and/or signals associated with other functions that enable the operation of the sensor 324. In examples, the sensor 324 may provide or receive clock, timing, enable, and/or other signals associated with operation of the sensor 324.

The sensor 324 (or elements thereof) also provides direction and distance data associated with advancement/retraction of the endoluminal instrument 306 to the external device. In one example, the sensor 324 may provide processed data (such as described above) associated with the direction/distance traveled by the endoluminal instrument 306 on a frame-by-frame basis. In other examples, the sensor 324 may aggregate the direction/distance data over a specified interval and provide the aggregated direction/distance data to the external device. For instance, elements of the sensor 324 may aggregate the direction/distance data and provide the data to the external device every 200 ms, 500 ms, 1000 ms, or on another interval. The provided data may include a numeric value for the aggregate linear distance traveled over the time interval. In one example, the direction data may be in the form of a plus or minus sign appended to the distance value (e.g., “+2.2 mm,” “−1.3 mm,” etc.), to indicate advancement or retraction of the endoluminal instrument, respectively. In other examples, the direction data may be provided in a different format or as separate data from the distance data.

In addition to the direction/distance data, the sensor 324 may also provide timing data to the external device. For example, the sensor 324 may provide time stamps or other timing information with the direction/distance data. In some examples, the sensor 324 may provide the sampling frame rate or similar information to the external device, which may use the sampling rate information to determine the time between the image frames.

In some examples, the sensor 324 may provide unprocessed data to the external device, such as data or signals acquired by the optical receiver 354. The external device may analyze the unprocessed data or signals to determine direction/distance traveled by the endoluminal instrument 306. For instance, in examples where the optical receiver 354 includes one or more phototransistors, photodiodes, or similar optically-sensitive elements, the sensor may provide analog or digital data from the optical receiver 354 directly to the external device.

As described above, the direction/distance and/or timing data may be provided to the external device via a wired connection. In other examples, the sensor 324 may include elements for wireless communication with the external device, such as through BLUETOOTH, WiFi, or other type of wireless technology. In examples where the sensor 324 is wirelessly connected to the external device, the sensor 324 may include a battery or other power source, which provides electrical power to elements of the sensor 324.

In some examples, the sensor 324 may also include a input element, such as a button 364 or other input element that allows for a clinician to interact with the sensor. For instance, selection of the button 364 may cause a linear distance being measured by the sensor to be reset or zeroed.

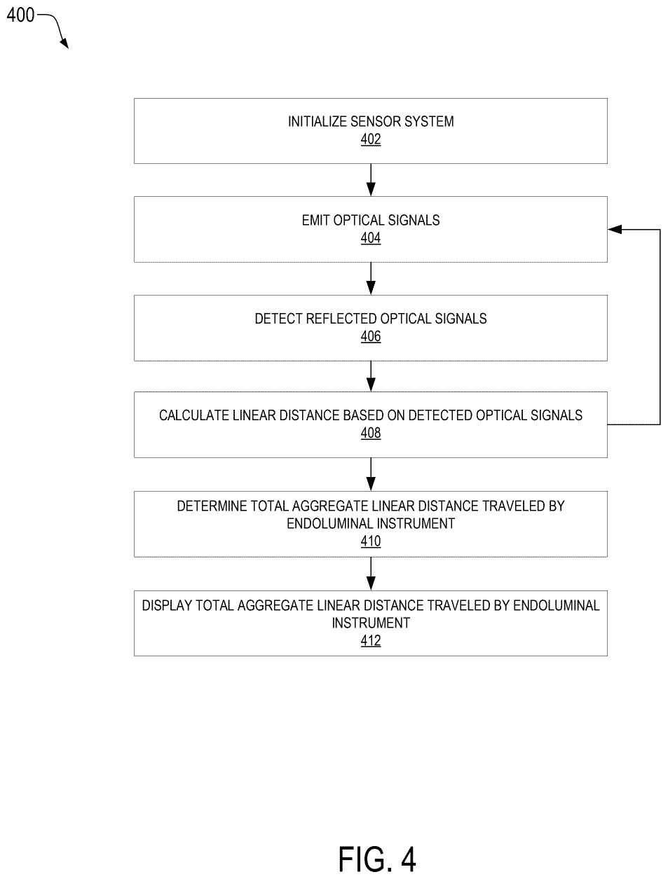

FIG. 4 depicts an example method 400 for determining the aggregate linear distance traveled by an endoluminal instrument (e.g., steerable endoscope 106) inserted into the airway through an airway access device (e.g., ETT 200A, OPA 200B, etc.). Example method 400 may be performed by an external device coupled to an airway access sensor (e.g., sensor 224) associated with the airway access device. For instance, example method 400 may be performed by a video laryngoscope (e.g., VL 102), which may also serve as a control device for the endoluminal instrument. In other examples, the method 400 and/or a portion of the operations thereof may be performed by the airway access sensor itself, such as by the processing unit(s) of the sensor.

In some examples, prior to method 400 being performed, the airway access device, including or coupled to the airway access sensor, is inserted and/or positioned in the airway of a patient. As described above, the airway access device may include an ETT, OPA, LMA, or other type of airway access device. In some examples, the airway access device may be inserted and positioned in the airway prior to insertion of the endoluminal instrument. For instance, an OPA may be positioned in the airway of a patient to maintain patency of the airway. An endoluminal instrument may then be inserted through the OPA and may provide video images of the airway.

In other examples, the airway access device may be inserted and positioned in the airway following insertion of the endoluminal instrument. In one example, an endoscope may be used to visualize a portion of the airway (e.g., the larynx) prior to intubation. An ETT may then be post-loaded over the endoscope, as described above.

At operation 402, the sensor system is initialized. The sensor system may include the external device, airway access sensor, and/or other associated devices (e.g., a computer, another medical device, etc.). Initialization may include establishing a connection between the airway access sensor to the external device. In examples where the airway access sensor is connected to the external device via wired connection (e.g., wire(s), cable(s), etc.) the detection of the sensor and/or the devices is performed via the wired connection that is established between the airway access sensor and the external device. The airway access sensor may receive electrical power from the external device and communication may be established through the wired connection.

In examples where the airway access sensor and external device are coupled via wireless connection (e.g., Bluetooth, WiFi, etc.), the airway access sensor and external device are powered on and wireless connection is established between the two devices. The wireless connection may be established according to protocols of the wireless technology.

Initializing the sensor system may also include initializing or zeroing an aggregate distance. In some examples, the external device may automatically initialize the aggregate linear distance when the airway access sensor is connected and/or powered on. In one example, the external device may automatically initialize the total aggregate linear distance to zero when the airway access sensor is powered on, regardless of whether the endoluminal instrument is present (e.g., detected by the airway access sensor) or not present (e.g., not detected by the airway access sensor).

In another example, the external device may automatically initialize the aggregate linear distance when the airway access sensor first detects insertion of an endoluminal instrument. For instance, an airway access device may be positioned in the airway and the distal tip of the endoluminal instrument may be inserted into the airway access device until the airway access sensor detects the distal tip of the endoluminal instrument. The external device may use the initial detection of the distal tip as an input that initializes the aggregate linear distance to zero.

In other examples, an input may be received from a clinician that sets (or resets) the aggregate linear distance of the endoluminal instrument to zero. For instance, a clinician may insert an endoluminal instrument into the airway access device until the endoluminal instrument is detected by the airway access sensor. The clinician may then provide an input to initialize the aggregate linear distance to zero, such as by pressing a physical button or tapping a virtual button on a touch-sensitive display of the external device. Setting the aggregate linear distance to zero may allow the clinician to set a baseline or reference position, from which further movement (advancement/retraction) of the endoluminal instrument may referenced. The clinician may provide the input at any time while using the endoluminal instrument. For example, the clinical may post-load an ETT over an endoscope during an intubation procedure. As the ETT is slid over the endoscope, the endoscope proximal end may pass, and be detected by, the airway access sensor. The clinician may position the ETT in the airway as desired to complete the intubation. Prior to retracting the endoscope from the ETT, the clinician may provide an input to initialize the total aggregate linear distance to zero. The external device may then aggregate the linear distance travelled by the endoscope as it is retracted (described below). The total aggregate linear distance observed during retraction may then be used in subsequent procedures where the endoscope or other instrument is inserted and advanced into the airway.

At operation 404, optical signals are emitted, such as light emitted from an optical emitter (e.g., LED, laser) of the sensor. The optical signals may illuminate the surface of the endoluminal instrument as the instrument passes by the sensor. The emitted light then reflects from the surface of the endoluminal instrument. At operation 406, the optical signals (e.g., light) that has reflected from the surface of the endoluminal instrument is detected. The optical signals may be detected by an optical receiver of the sensor. In some examples, detecting the optical signals may include capturing an image.

At operation 408, based on the detected optical signal, a linear distance that that the endoluminal instrument has moved passed the sensor is calculated. The linear distance may be calculated and/or determined based on any of the processes discussed above, such as comparison of the captured image to a previously captured image. The distance calculated may be a positive or negative distance indicating a direction of travel by the endoluminal instrument. The calculated linear distance data may include incremental distances travelled by the endoluminal instrument as determined by the airway access sensor on a frame-by-frame basis. As an example, the airway access sensor may process subsequent image frames and determine that between image frames the endoluminal instrument was advanced into the airway by 0.2 mm.

At operation 410, the total aggregate linear distance traveled by the endoluminal instrument is determined. The external device uses the linear distance data received at operation 408 to determine the total aggregate linear distance. In examples where the airway access sensor provides linear distance data on a frame-by-frame basis, the external device may determine the total aggregate linear distance traveled by the endoluminal instrument by maintaining a cumulative total of each of the distances traveled between image frames. As an example, linear distance data may indicate that the endoluminal instrument advanced into the airway by 0.2 mm between a first and second image frame, advanced by 0.1 mm between the second and a third image frame, retracted by 0.1 mm between the third and a fourth image frame, and so on. After receiving distance data associated with the third and fourth image frames, the total aggregate linear distance traveled by the endoluminal instrument is 0.2 mm towards or into the airway. The linear distance traveled as additional linear distance data is received.

The determinations or calculations of the incremental linear distances and/or the aggregate linear distance may be performed by the sensor itself (e.g., by processing and memory elements of the sensor) and/or by the external device (e.g., controller, video laryngoscope). For instance, the airway access sensor may provide analog or digital data directly from photo-sensitive elements of the airway access sensor to the external device. The external device may process the received data for further analysis, such as by filtering, amplifying, digitizing (if needed), storing, and/or performing other processing. In other examples, the incremental linear distances are calculated by the sensor and then transmitted to the external device. In yet other examples, the incremental and aggregated linear distances are calculated by the sensor and the aggregate linear distances are then transmitted to the external device in intervals or as the aggregate linear distance is updated. In some examples, the external device receives linear distance data aggregated by the airway access sensor over a time interval, and the external device determines the total aggregate linear distance by maintaining a cumulative total of each of the aggregated distances traveled during the time intervals. As another example, the airway access sensor may measure and aggregate distance traveled over 200 ms time intervals and provide aggregated linear distance data to the external device. A first measurement may indicate advancement of the endoluminal instrument by 0.4 mm, a second measurement may indicate advancement by 0.4 mm, a third measurement may indicate retraction by 0.1 mm, and so on. After receiving the third measurement, the external device determines that the total aggregate linear distance traveled by the endoluminal instrument is 0.7 mm toward or into the airway, over the 600 ms total interval. The external device continues to accumulate the linear distance traveled as additional linear distance data is received.

At operation 412, the total aggregate linear distance traveled by the endoluminal instrument is displayed or caused to be displayed. In one example, the aggregate linear distance may be displayed on a display of the external device, such as a display of a controller and/or a display of the video laryngoscope. In other examples, the distance may be displayed on another display associated with the sensor system. For instance, the external device may provide the distance to an alternate device, such as a computer, monitor, another medical device, etc., and the alternate device may display the total aggregate linear distance traveled by the endoluminal instrument.

In another example, the total aggregate linear distance may be displayed on a display associated with an augmented visualization system. For instance, the linear distance data, IMU data, endoscope camera data, and/or other data may be provided on a specialized display (e.g., a headset or other type of display) in an integrated viewing format. For instance, the augmented reality system may display a position of the endoscope in the body as an overlay of the patient and/or another augmented reality view. The displayed position may be based on the total aggregate linear distance that has been traveled. The displayed position may also be based on other factors, such as IMU measurements and/or images captured by the endoscope. For instance, during an initial navigation of the endoscope through the airway, the image data acquired by the endoscope camera may be used to perform image recognition of anatomical structures and/or regions within the airway (e.g., trachea, carina, first generation of the bronchial tree, etc.). During subsequent navigation of the endoscope through the airway, the image data may be used to coarsely identify the general region of the airway (or other segment of the body) and the linear distance data may be used to provide more precise position information within the identified region.

In further examples, the sensor and/or the airway access device may itself include a small display that displays the linear distance of the endoluminal tool. Operations 404-412 may then be repeated as the endoluminal tool continues to pass by the sensor. Thus, the linear distance that the endoluminal tool travels may continue to be measured and conveyed to the clinician

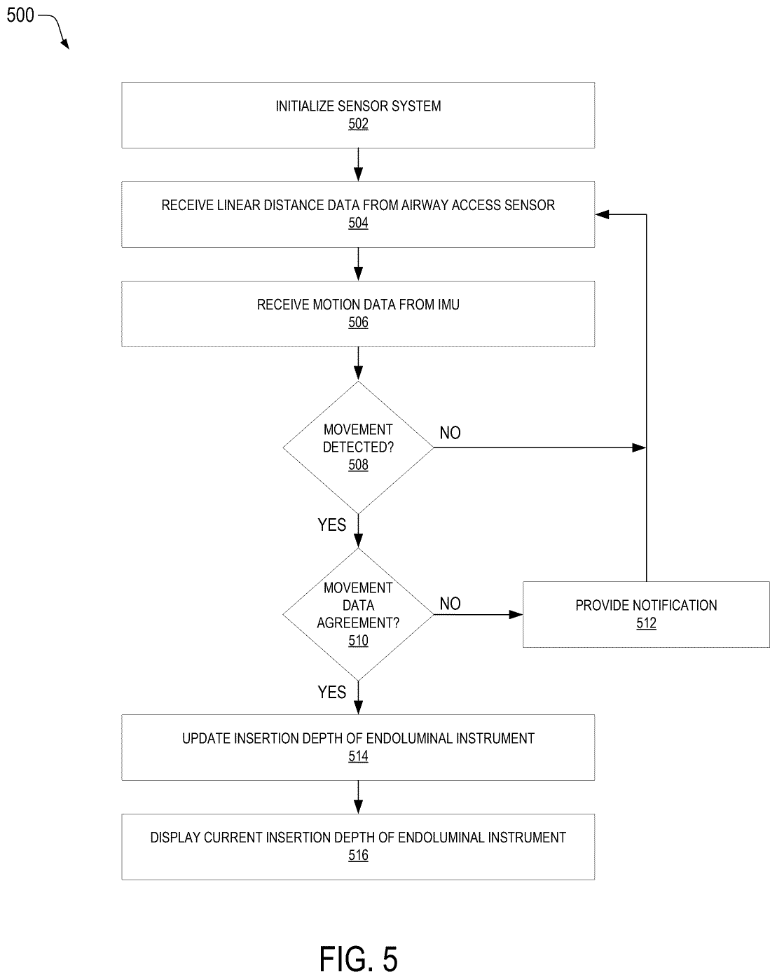

FIG. 5 depicts an example method 500 for using motion data to verify insertion depth of an endoluminal instrument. Example method 500 may, in some examples, be performed by an external device coupled to an airway access sensor associated with an airway access device. For instance, example method 500 may be performed by a VL (e.g., VL 102), which may also serve as a control device for an endoluminal instrument (e.g., endoscope 106). In other examples, the endoluminal instrument may be controlled by a first device (e.g., a control device) and the airway access sensor may be connected to a second device (e.g., a separate external device). In such examples, the first and second devices may be communicatively coupled, and data may be exchanged as needed, so that example method 500 may be performed as described herein. operations of method 500 may also be performed by the sensor itself. To facilitate description, example method 500 is described from the perspective of a single external device, such as a VL, which is connected to both the endoluminal instrument and airway access sensor.

At operation 502, the sensor system is initialized. The sensor system may include the external device, airway access sensor, motion sensor associated with the endoluminal instrument, and/or other associated devices. The motion sensor is described herein as an IMU, but in other examples may be another type of motion sensor capable of provided motion data per the operations described below.

Initialization includes connecting and powering on the external device and airway access sensor as described for operation 402 of example method 400. The endoluminal instrument is also connected to the external device, such as by plugging the endoluminal instrument directly into the external device. In some examples, the endoluminal instrument may be connected to an interface device (e.g., detachable cartridge 104), which in turn is connected to the external device. When the endoluminal instrument is connected to the external device, the endoluminal instrument receives electrical power and provides video image and IMU motion data to the external device.

The external device may establish synchronization between the endoluminal instrument and the airway access sensor, such that linear distance data received from the airway access sensor and motion data received from the IMU may be compared to one another within a common timing structure. In one example, the external device may provide a clock or other timing signal to the endoluminal instrument and airway access sensor or may request and receive linear distance data from the endoluminal instrument and airway access sensor according to an established (known) timing arrangement.

As described above, during initialization (or at other times), a clinician may configure operation of the sensor system. For example, the clinician may establish a baseline or reference position, such as by initializing the linear distance travelled by the endoluminal instrument to zero. The clinician may also configure aggregation timing intervals, image frame rates, thresholds, etc.

At operation 504, linear distance data is calculated and/or received from the airway access sensor, such as described above in example method 400. In some examples, the linear distance data may include measurements of the linear distance traveled by the endoluminal instrument on a frame-by-frame basis. In other examples the linear distance data may include linear distance measurements aggregated over a time interval (e.g., 200 ms, 500 ms, etc.), or other types of linear distance measurements.

At operation 506, motion data is received from the IMU. The received motion data includes data measured for a plurality of orthogonal motion-sensitive axes. For example, the IMU may provide motion data as measured for an x-axis, y-axis, and z-axis, or may provide motion data corresponding to another axis system provided by the IMU. The motion data may include position, velocity, acceleration, angular velocity, angular acceleration, and/or other variables associated with the position, orientation, and/or movement of the endoluminal instrument.

In some examples, the IMU is located in a steerable distal tip of the endoluminal instrument (e.g., steerable tip 118 of endoscope 106). In such examples, the motion data is be associated with the position, orientation, and/or movement of the steerable tip of the endoluminal instrument. As the steerable tip is articulated and the endoluminal instrument advanced or retracted, the motion sensed by the IMU may be projected onto different motion-sensitive axes. For instance, if the steerable tip of the endoluminal instrument is articulated by 90 degrees, motion that was previously sensed along one motion-sensitive axis (e.g., the x-axis), may be projected onto an orthogonal motion-sensitive axis (e.g., the y-axis or z-axis). Accordingly, advancement or retraction of the endoluminal instrument may be determined by accounting for the orientation of the steerable tip.

At operation 508, a determination is made as to whether linear distance data from the airway access sensor and/or motion data from the IMU indicates that the endoluminal instrument has moved from a previous position. For example, linear distance data from the airway access sensor indicates movement if the endoluminal instrument has been advanced into, or retracted from, the airway by a distance measurable by the airway access sensor. In addition, motion data provided by the IMU may indicate movement of the endoluminal instrument in any of a variety of ways. For instance, the IMU may detect measurable acceleration along one or more of the motion-sensitive axes of the IMU. In another example, the IMU may determine the velocity and/or position of the endoluminal instrument, such as by performing mathematical integration of the acceleration data. The velocity and/or position data may indicate movement of the endoluminal instrument. In other examples, other motion data provided by the IMU may be used to determine movement of the endoluminal instrument.

If either the linear distance data provided by the airway access sensor or the motion data provided by the IMU indicate that no movement of the endoluminal instrument has occurred, flow proceeds “NO” and returns to operation 504. The external device may continue to receive linear distance data and motion data for determining movement of the endoluminal instrument. If the linear distance data provided by the airway access sensor and/or the motion data provided by the IMU indicate that movement of the endoluminal instrument has occurred, flow proceeds “YES” to operation 510.

At operation 510, a determination is made as to whether the linear distance data provided by the airway access sensor is commensurate with or matches (within a threshold) the motion data provided by the IMU, where both sets of data indicate approximately the same degree of relative movement by the endoluminal instrument.

In one example, the airway access sensor may provide linear distance data indicating advancement of the endoluminal instrument by a first distance over an interval of time. The IMU may provide motion data indicating advancement of the endoluminal instrument by a second distance over the same interval of time. The external device may compare the first distance to the second distance and may use a threshold to determine whether the first and second distances are commensurate. In some examples, the threshold may be specified in terms of a percent difference. For example, the external device may determine that the first and second distances are commensurate if the second distance is within 5% of the first distance, 10% of the first distance, 15% of the first distance, or is within another measure of percent difference from the first distance. In other examples, the threshold may be specified in terms of absolute difference in distances. For example, the external device may determine that the first and second distances are commensurate if the second distance is within 0.2 mm of the first distance, 0.5 mm of the first distance, or is within some other distance of the first distance. The threshold may be specified by the clinician, such as during initialization of the sensor system, as described for operation 502.

In some examples, the external device may determine the second distance by analyzing the received IMU data. For example, the external device may analyze position data received from the IMU to determine the positional changes of the endoluminal instrument over the time interval. The positional changes may be further used to determine linear distance traveled by the endoluminal instrument, as measured by the IMU. In another example, the external device may receive acceleration and/or velocity data from the IMU and may perform mathematical integration to determine positional changes of the endoluminal instrument over the time interval.

In still other examples, the external device may analyze the linear distance data received from the airway access sensor in order to compare the airway access sensor data to the motion data received from the IMU. For example, the external device may perform mathematical differentiation of the linear distance data to determine velocity and/or acceleration of the endoluminal instrument. The external device may then compare the computed velocity/acceleration derived from the airway access sensor data to the velocity/acceleration data received from the IMU. As described above, the external device may compare the data by applying one or more thresholds.

In examples where the external device determines that the airway access sensor data and IMU data do not agree on the amount of movement of the endoluminal instrument, flow proceeds “NO” to operation 512. At operation 512, the external device provides a notification to the clinician that the movement data do not agree. The notification may be in the form of an audible alert, such as may be provided by a speaker associated with the external device. In some examples, the external device may provide a visual alert, such as by illuminating an indicator light (e.g., an LED or bulb), or by providing a visual alert on a display associated with the external device. The notification may indicate that the endoluminal tool is caught or otherwise prevented from moving forward at the distal tip of the endoluminal tool.

Additionally or alternatively, the external device may be capable of providing haptic feedback to the clinician as a form of notification. For instance, a handle or other portion of the external device may include a vibratory motor or other haptic generator that provides a tactile sensation, such as vibrations, one or more taps, or other type of haptic feedback.

In response to the notification, the clinician may reduce the force being applied to the endoluminal instrument, may stop advancing the endoluminal instrument, or may retract the endoluminal instrument to prevent damage to the tissues of the airway. In some examples, the clinician may alter how the endoluminal instrument is advanced through the airway, such as by using video images and providing steering control to advance the endoluminal instrument along a different route. Following the notification, the external device continues to receive linear distance and motion data (per operations 504 and 506) and continues performing other operations of the example method 500.

Returning to operation 510, in examples where the external device determines that the airway access sensor data and IMU data agree on the amount of movement of the endoluminal instrument, flow proceeds “YES” to operation 514. At operation 514, the external device updates the insertion depth of the endoluminal instrument. The insertion depth may be based on the aggregated linear data from the airway access sensor alone. In other examples, the insertion depth is based on a combination of the aggregated linear data from the airway access sensor and the position data generated from the IMU signals from the endoluminal instrument. Such examples allow for the accounting of distal tip articulation and movement within the body. In yet other examples, the insertion depth is based on the position data from the IMU signals.

At operation 516, the current insertion depth of the endoluminal instrument is displayed. In examples, the current insertion depth may be displayed on a display associated with a VL, other control device, other medical device, and/or other type of device or monitor. In some examples the current insertion depth may be displayed on a display associated with an augmented visualization system, as described above. For instance the augmented visualization system may display the current position, IMU data, endoscope camera image data, and/or other data on a common display (e.g., a headset) in an integrated viewing format.

Those skilled in the art will recognize that the methods and systems of the present disclosure may be implemented in many manners and as such are not to be limited by the foregoing aspects and examples. In other words, functional elements being performed by a single or multiple components. In this regard, any number of the features of the different aspects described herein may be combined into single or multiple aspects, and alternate aspects having fewer than or more than all of the features herein described are possible. Functionality may also be, in whole or in part, distributed among multiple components, in manners now known or to become known.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurement techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. An airway access device comprising:
a body;
a lumen defined by the body; and
an optical sensor, coupled to the body, comprising:
an optical emitter that emits light towards an endoluminal instrument that passes through the lumen;
an optical receiver that detects light reflected from the endoluminal instrument; and
a processing unit that calculates a linear distance that the endoluminal instrument has traveled.

2. The airway access device of claim 1, wherein the airway access device is an endotracheal tube, the body defines a tubing connector, and the optical sensor is coupled to the tubing connector.

3. The airway access device of claim 1, wherein the airway access device is an oropharyngeal airway, the body defines a bite block, and the optical sensor is coupled to the bite block.

4. The airway access device of claim 1, further comprising a retaining guide that retains the endoluminal instrument in proximity with the optical sensor.

5. The airway access device of claim 4, wherein the retaining guide is a spring clip that is biased towards the optical sensor.

6. The airway access device of claim 1, wherein calculating a linear distance that the endoluminal instrument has traveled comprises:
comparing a first image captured by the optical receiver with a second image captured by the optical receiver to determine an incremental linear distance; and
aggregating the linear distance with prior incremental linear distances to generate the aggregate linear distance that the endoluminal instrument has traveled.

7. The airway access device of claim 1, wherein the processing unit further causes the optical sensor to transmit the linear distance to an external device.

8. An airway access system comprising:
an endoluminal instrument controller that is coupled to an endoluminal instrument;
an airway access sensor, coupled to an airway access device, in communication with the endoluminal instrument controller, wherein the airway access sensor:
measures a linear distance that the endoluminal instrument passes by the airway access; and
transmits the linear distance to the endoluminal instrument controller.

9. The airway access system of claim 8, wherein the endoluminal instrument controller is a video laryngoscope and the endoluminal instrument is one of an endoscope or an introducer.

10. The airway access system of claim 8, wherein the airway access sensor is integrated into a tubing connector.

11. The airway access system of claim 8, wherein the endoluminal instrument comprises a motion sensor in a distal tip of the endoluminal instrument, and the endoluminal instrument controller:
receives motion data from the motion sensor;
calculates a distance travelled by the endoluminal instrument based on the motion data from the motion sensor;
compares the distance travelled to the linear distance from the airway access sensor; and
based on the comparison, generating a notification.

12. A method for measuring a linear distance that an endoluminal instrument travels into a body, the method comprising:
detecting, by an optical receiver of an airway access sensor, light reflected from the endoluminal instrument at multiple points in time;
based on the detected light, calculating an incremental distance travelled by the endoluminal instrument for the multiple points in time;
aggregating the incremental distances to form an aggregate linear distance; and
displaying the aggregate linear distance as the endoluminal instrument travels into or out of the body.

13. The method of claim 12, wherein the airway access sensor is coupled to an airway access device.

14. The method of claim 12, further comprising:
receiving motion data from a motion sensor in a distal tip of the endoluminal instrument;
calculating a distance travelled by the endoluminal instrument based on the motion data;
comparing the distance travelled based on the motion data with the aggregate linear distance; and
based on the comparison, displaying a notification.

15. The method of claim 14, wherein calculating the incremental distances is performed by the airway access sensor and calculating a distance travelled by the endoluminal instrument based on the motion data is performed by an endoluminal instrument controller.
